(19) United States
(12) Patent Application Publication
(10) Pub. No.: US 2006/0177399 A1
(43) Pub. Date: Aug. 10, 2006

(54) Pressurized device for hair fiber styling and use of the same for hair fiber styling and/or hair

(76) Inventors: Herve Jourdan, Saint-Cyr (FR); Regis Beitone, Paris (FR); Francois Le Bourhis, Aubervilliers (FR); Pierre-Andre Lasserre, Coubron (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/274,304

(22) Filed: Nov. 16, 2005

Related U.S. Application Data
(60) Provisional application No. 60/646,611, filed on Jan. 26, 2005.

(30) Foreign Application Priority Data
Nov. 16, 2004 (FR) 04 52646

(51) Int. Cl. A61K 8/00

(52) U.S. Cl. 424/70.1

(57) ABSTRACT

The present invention relates to a pressurized device for hair fiber styling and to its use for hair fiber styling and/or care. The pressurized device, especially an aerosol, comprises a container, the wall of which is made at least partly of a mixture comprising at least one polyethylene naphthalate and at least one polyethylene terephthalate, a cosmetic composition comprising an aqueous phase comprising at least one foaming agent; and an organic phase comprising at least one propellant selected from halogenated or not halogenated hydrocarbons and mixtures thereof, said device being able to withstand high temperatures.

This application claims benefit of U.S. Provisional Application No. 60/646,611, filed Jan. 26, 2005, the contents of which are incorporated herein by reference. This application also claims benefit of priority under 35 U.S.C. § 119 to French Patent Application No. 04 52646, filed Nov. 16, 2004, the contents of which are also incorporated by reference.

The present invention relates to a pressurized device comprising a cosmetic composition and to its use for hair styling and/or hair fiber care.

Cosmetic compositions that are packaged in aerosol containers are usually pressurized compositions to be sprayed by means of a propellant in metal cans, such as aluminium or tinplate.

Such pressurized devices are often stored before being marketed in warehouses where the storage time may last for up to several months.

When stored in such conditions, these pressurized devices may undergo high temperatures.

But these pressurized devices, above all when made of plastic, suffer as a drawback from not withstanding high temperatures, whereupon they may deform or even burst.

It is therefore an object of the invention to provide a pressurized device, especially an aerosol, comprising a cosmetic composition, that withstands high temperatures and that does not burst or deform, especially during long storage cycles.

The applicant indeed surprisingly discovered that it was possible to counteract the drawbacks of the prior art and to satisfy the objects mentioned hereabove by making a pressurized device, especially an aerosol, comprising a container, the wall of which is made at least of polyethylene naphthalate, an aqueous two-phase cosmetic composition comprising at least one propellant selected from hydrocarbons and mixtures thereof, and at least one foaming agent, said composition being released by means of a diffuser as a foam.

So, it is an object of the present invention to provide a pressurized container, especially an aerosol, comprising:

a) a container, the wall of which is made at least partly of a mixture of at least one polyethylene naphthalate and at least one polyethylene terephthalate,

b) a cosmetic composition comprising:
an aqueous phase comprising at least one foaming agent; and
an organic phase comprising at least one propellant selected from halogenated or not halogenated hydrocarbons and mixtures thereof,
said device being able to withstand high temperatures.

As used herein, a device that withstands high temperatures means a device that does not explode when raised to and maintained for two hours, at a temperature of 80° C.

Preferably, the container according to the invention comprises, as compared to the polymer total weight, from 60% to 95%, more preferably 90% polyethylene naphthalate and from 5% to 40%, more preferably 10% polyethylene terephthalate.

Preferably, device's container according to the invention is transparent or translucent.

As used herein, transparent or translucent containers mean containers through the wall of which the cosmetic composition may be seen.

Preferably, the composition contained in the device according to the invention is transparent or translucent.

As used herein, transparent or translucent compositions mean compositions which turbidity, as measured according to the method described hereafter, is lower than 800 NTU (Nephelometric Turbidity Units) and preferably lower than 500 NTU.

Turbidity measure is conducted by means of a turbidimeter model 2100 P marketed by HACH™ at ambient temperature (between 20 and 25° C.). Tubes used to measure have following references: AR 397 A cat 24347-06. Calibrating the device occurs using formazine suspensions with different concentrations.

As used herein, a foam means a mixture comprising the cosmetic composition and the propellant, which density as compared to water, as measured at 25° C. and at atmospheric pressure (1.013 bar), may reach up to 0.1.

As used herein, a two-phase composition means a composition comprising an aqueous phase and an organic phase, that are not miscible with each other.

Preferably, at least one of both cosmetic composition phases is transparent.

Should the container according to the invention be made of a polyethylene naphthalate-polyethylene terephthalate mixture, such mixture comprises at least 50%, preferably at least 75% by weight polyethylene naphthalate as compared to the polymer mixture total weight.

As previously stated, the composition according to the invention comprises at least one foaming agent selected from the group consisting of cationic, anionic, non ionic, amphoteric or zwitterionic surfactants and cationic, anionic, non ionic, amphoteric or zwitterionic polymers.

As used herein, a foaming agent means an agent that produces a foam when in presence of a combination comprising water, a propellant and a suitable release device.

Surfactants to be suitably used for carrying out the present invention are especially as follows:

(i) Anionic Surfactant(s):

Their nature is not substantially critical in the context of the present invention.

So, examples of anionic surfactants that may be used, alone or in a mixture, in the context of the present invention especially comprise (without being limited to) salts (especially alkaline salts, especially sodium salts, ammonium salts, amine salts, amino alcohols salts or magnesium salts) of following compounds: alkyl sulfates, alkyl ether sulfates, alkyl amidoether sulfates, alkylaryl polyether sulfates, monoglyceride sulfates; alkyl sulfonates, alkyl phosphates, alkyl amide sulfonates, alkylaryl sulfonates, α-olefin sulfonates, paraffin sulfonates; alkyl sulfosuccinates, alkyl ether sulfosuccinates, alkyl amide sulfosuccinates; alkyl sulfosuccinamates; alkyl sulfoacetates; alkyl ether phosphates; acyl sarcosinates; acyl isethionates and N-acyl taurates, the alkyl or acyl radical of all these different compounds containing preferably from 12 to 20 carbon atoms, and the aryl radical preferably representing a phenyl or a benzyl moiety. There are also amongst these further usable anionic surfactants, fatty acid salts, such as oleic, ricinoleic, palmitic and stearic acid salts, coconut oil or hydrogenated coconut oil acids; acyl lactylates the acyl radical of which contains from 8 to 20 carbon atoms. Weakly anionic surfactants may also be used, such as alkyl D-galactoside uronic acids and salts thereof, as well as polyoxyalkylene carboxylic ether acids and salts thereof, especially those containing from 2 to 50 ethylene oxide moieties, and mixtures thereof. Anionic surfactants of the polyoxyalkylene carboxylic ether acid or salt type are especially those represented by following formula (1):
R₁—(OC₂H₄)ₙ—OCH₂COOA (1)

wherein:

R₁ represents an alkyl, alkylamido or alkaryl moiety, and n is an integer or a decimal (average) varying from 2 to 24 and preferably from 3 to 10, the alkyl radical having from about 6 to 20 carbon atoms, and aryl representing preferably a phenyl group,

A represents H, ammonium, Na, K, Li, Mg or a monoethanolamine or triethanolamine residue. Mixtures of compounds of formula (1) may also be used, especially mixtures wherein R₁ moieties are different.

Compounds of formula (1) are for example marketed by CHEM Y under the trade names AKYPOS (NP40, NP70, OP40, OP80, RLM25, RLM38, RLMQ 38 NV, RLM 45, RLM 45 NV, RLM 100, RLM 100 NV, RO 20, RO 90, RCS 60, RS 60, RS 100, RO 50) or by SANDOZ under the trade names SANDOPAN (DTC Acid, DTC).

(ii) Non ionic Surfactant(s):

Non ionic surfactants are also compounds that are well known per se (see especially “Handbook of Surfactants” M. R. PORTER, Blackie & Son Editors (Glasgow and London), 1991. pp 116-178) and their nature is not critical in the context of the present invention. They therefore may be especially selected from the (non limitative) group consisting of polyethoxylated, polypropoxylated or polyglycerolated fatty alcohols, polyethoxylated, polypropoxylated or polyglycerolated fatty alpha diols, polyethoxylated, polypropoxylated or polyglycerolated fatty alkyl phenols and polyethoxylated, polypropoxylated or polyglycerolated fatty acids, all of which having a fatty chain containing for example from 8 to 18 carbon atoms, with a number of ethylene oxide moieties or propylene oxide moieties especially ranging from to 2 to 50 and a number of glycerol moieties especially ranging from 2 to 30. Ethylene oxide and propylene oxide copolymers may also be mentioned, as well as ethylene oxide and propylene oxide condensation products on fatty alcohols; polyethoxylated fatty amides having preferably from 2 to 30 moles ethylene oxide, polyglycerolated fatty amides containing in average from 1 to 5 glycerol moieties and especially from 1.5 to 4; sorbitan oxyethylenated fatty acid esters having from 2 to 30 moles ethylene oxide; sucrose fatty acid esters, polyethylene glycol fatty acid esters, alkyl polyglycosides, N-alkyl glucamine derivatives, amine oxides, such as (C₁₀-C₁₄)alkyl amine oxides or N-acyl aminopropyl morpholine oxides.

(iii) Amphoteric or zwitterionic Surfactant(s):

Amphoteric or zwitterionic surfactants, the nature of which isn't critical in the context of the present invention may especially include (without being limited to) aliphatic secondary or tertiary amine derivatives, where the aliphatic radical is a linear or branched chain containing from 8 to 18 carbon atoms and at least one hydrosolubilizing anionic group (for example carboxylate, sulfonate, sulfate, phosphate or phosphonate); (C₈-C₂₀)alkyl betaines, sulfobetaines, (C₈-C₂₀)alkyl (C₁-C₆)amidoalkyl betaines or (C₈-C₂₀)alkyl (C₁-C₆)amidoalkyl sulfobetaines may also be mentioned.

Amongst amine derivatives, there are those products marketed under the trade name MIRANOL, such as described in the American patents U.S. Pat. No. 2,528,378 and U.S. Pat. No. 2,781,354 and categorized in the CTFA dictionary, third edition, 1982, under the names “amphocarboxyglycinates” and “amphocarboxypropionates” and having following structure, respectively:
R₂—CONHCH₂CH₂—N+(R₃)(R₄)(CH₂COO—) (2)

wherein: R₂ represents an alkyl radical of a R₂—COOH acid present in hydrolyzed coconut oil, a heptyl, nonyl or undecyl radical, R₃ represents a β-hydroxyethyl moiety and R₄ a carboxymethyl moiety;
and
   R₂′—CONHCH₂CH₂—N(B)(C) (3)
wherein:

B represents —CH₂CH₂OX′, C represents —(CH₂)z—Y′, with z=1 or 2,

X′ represents the —CH₂CH₂—COOH moiety or a hydrogen atom,

Y′ represents —COOH or the —CH₂—CHOH—SO₃H radical,

R₂′ represents an alkyl radical of a R₉—COOH acid present in coconut oil or in hydrolyzed linseed oil, an alkyl radical, especially C₇, C₉, C₁₁ or C₁₃, a C₁₇alkyl radical and its iso form, an unsaturated C₁₇ radical.

Cocoamphocarboxyglycinate is a suitable example, such as is marketed under the trade name MIRANOL C2M concentrated, by MIRANOL.

(iv) Cationic Surfactants:

Cationic surfactants may particularly include (but without being limited to) primary, secondary or tertiary fatty amine salts, optionally polyoxyalkylenated; quaternary ammonium salts such tetraalkyl ammonium, alkylamidoalkyl trialkylammonium, trialkylbenzyl ammonium, trialkylhydroxyalkyl ammonium or alkyl pyridinium chlorides and bromides; imidazoline derivatives; or amine oxides of cationic type.

Any polymer known in the hair care field having foaming properties, as well as mixtures comprising many of such polymers, may also be used in the present invention. Polymers are ordinarily classifed into cationic, anionic, non ionic, amphoteric or zwitterionic polymers.

Cationic polymers that can be used according to the invention are preferably selected from polymers containing primary, secondary, tertiary and/or quaternary amine moieties belonging to the polymeric chain or directly bound to it, and having a molecular weight ranging from 500 to about 5 000 000 and preferably from 1000 to 3 000 000.

Amongst these polymers, the following cationic polymers may be particularly mentioned:

(1) acrylic or methacrylic ester or amide-derived homopolymers or copolymers containing at least one of the following units: 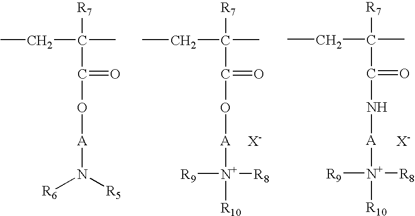
wherein:

R₅ and R₆ each represent a hydrogen atom or a C₁₋₆ alkyl group,

R₇ represents a hydrogen atom or a CH₃ radical.

R₈, R₉ and R₁₀, which may be the same or different, each represent a C₁₋₁₈ alkyl group or a benzyl radical,

A is a linear or branched C₁₋₆ alkyl group or a C₁₋₄ hydroxyalkyl group and

X⁻ represents a methosulfate or halide anion, such as a chloride or bromide ion.

Moreover, family (1) copolymers comprise one or more unit(s) derived from comonomers that may be selected from the family of acrylamides, methacrylamides, diacetone acrylamides, acrylamides and methacrylamides substituted on nitrogen atom by lower alkyl groups, acrylic or methacrylic acids or esters thereof, vinyl lactames, such as vinyl pyrrolidone or vinyl caprolactame, and vinyl esters.

Vinyl lactame units preferred according to the invention are those that comprise units of formula:

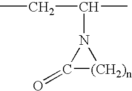

wherein n independently represents 3, 4 or 5.

Advantageously, vinyl lactame unit containing polymers are copolymers or may also comprise units of formula

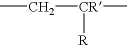

wherein

R independently represents a carboxy radical, a carbalcoxy radical, an acyloxy radical, where the alkoxy moiety of the carbalcoxy radical may be substituted by at least one hydroxy, amino, alkylamino or dialkylamino radical or by an aryl radical, especially phenyl, optionally substituted by at least one alkyl radical, and

R′ represents a hydrogen atom or an alkyl radical.

As used herein, alkyl means preferably linear or branched C₁-C₁₀ alkyl radicals, more preferably C₁-C₄ alkyl radicals, especially methyl, ethyl, n-propyl, i-propyl, n-butyl and t-butyl radicals.

As used herein, acyl means preferably, acyl radicals, the alkyl residue of which is a linear or branched C₁-C₁₀ alkyl, especially acetyl or propionyl radicals.

Vinyl lactame unit containing polymers according to the invention are preferably non ionic or weakly cationic polymers. They are especially described in the American patents U.S. Pat. No. 3,770,683, U.S. Pat. No. 3,929,735, U.S. Pat. No. 4,521,504, U.S. Pat. No. 5,158,762, U.S. Pat. No. 5,506,315 and in the patent applications WO 94/121148, WO 96/06592 and WO 96/10593.

So, family (1) copolymers may be mentioned as follows:

acrylamide and dimethylaminoethyl methacrylate copolymers quaternized with dimethyl sulfate or with a dimethyl halide, such as the product marketed under the trade name HERCOFLOC® by HERCULES,

acrylamide and methacryloyl oxyethyltrimethylammonium chloride copolymers described for example in the patent application EP-A-080976 and marketed under the trade name BINA QUAT® P 100 by CIBA GEIGY,

acrylamide and methacryloyloxyethyl trimethylammonium methosulfate copolymer marketed under the trade name RETEN® by HERCULES,

vinyl pyrrolidone/dialkylaminoalkyl acrylate or methacrylate copolymers quaternized or not, such as the products marketed under the trade name GAFQUAT® by ISP, as for example GAF-QUAT® 734 or GAFQUAT® 755, or non quaternized polyvinyl pyrrolidone/diaminoethyl methacrylate copolymers called COPOLYMER® 845, 958 and 937. These polymers are described in detail in the French patent applications FR 2,077,143 and FR 2,393,573,

dimethylaminoethyl methacrylate/vinyl caprolactame/vinyl pyrrolidone terpolymers, such as the product marketed under the trade name GAFFIX VC 713 by ISP, and

quaternized vinyl pyrrolidone/dimethylaminopropyl methacrylamide copolymer, such as the product marketed under the trade name GAFQUAT® HS 100 by ISP.

(2) quaternized polysaccharides more particularly described in the American patents U.S. Pat. Nos. 3,589,578 and 4,031,307, such as trialkylammonium cationic moiety containing guar gums.

Such products are especially marketed under the trade names JAGUAR® C13 S, JAGUAR® C15 and JAGUAR® C17 by MEYHALL.

(3) vinyl pyrrolidone and vinyl imidazole quaternary copolymers such as the products marketed by BASF under the trade name LUVIQUAT® TFC,

(4) chitosans or salts thereof, especially chitosan acetate, lactate, glutamate, gluconate or pyrrolidone carboxylate.

Chitosan with a 90.5% by weight deacetylation rate marketed under the trade name KYTAN BRUT STANDARD® by ABER TECHNOLOGIES may be mentioned, as well as chitosan pyrrolidone carboxylate marketed under the trade name KYTAMER® PC by AMERCHOL.

(5) cationic cellulose derivatives, such as cellulose copolymers or cellulose derivatives grafted with a water-soluble monomer containing a quaternary ammonium group and especially described in the American patent U.S. Pat. No. 4,131,576, such as hydroxyalkylcelluloses, such as hydroxymethylhydroxyethyl- or hydroxypropylcelluloses grafted especially with a methacryloyloxyethyl trimethylammonium salt, a methacrylamidopropyl trimethylammonium salt or a dimethyldiallylammonium salt.

The products on the market corresponding to such a definition are more particularly the products marketed under the trade names CELQUAT® L 200 and CELQUAT® H 100 by NATIONAL STARCH.

Anionic polymers that are usually used are polymers containing carboxylic, sulfonic or phosphoric acid derived moieties and having a weight average molecular weight ranging from about 500 to 5 000 000.

Carboxylic acid moieties are provided by unsaturated monomers containing one or two carboxylic acid functionalities, such as those corresponding to formula (4): 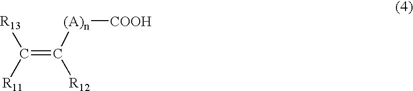
wherein n is an integer between 0 and 10, A represents a methylene moiety, optionally bound to the carbon atom of the unsaturated moiety or of the adjacent methylene moiety, when n is more than 1, through a heteroatom, such as oxygen or sulfur, R₁₃ represents a hydrogen atom, a phenyl or benzyl moiety, R₁₁ represents a hydrogen atom, a lower alkyl or a carboxyl moiety, and R₁₂ represents a hydrogen atom, a lower alkyl moiety, a-CH₂—COOH, phenyl or benzyl moiety.

In the aforementioned formula, a lower alkyl radical preferably means a moiety having from 1 to 4 carbon atoms and especially a methyl or ethyl group.

Preferred carboxylated anionic polymers according to the invention include:

A) homo- or copolymers of acrylic or methacrylic acid or salts thereof and especially the products marketed under the trade names VERSICOL® E or K by ALLIED COLLOID, and under the trade name ULTRAHOLD® by BASF; acrylic acid and acrylamide copolymers marketed as sodium salt under the trade names RETEN® 421, 423 or 425 by HERCULES; polyhydroxycarboxylic acid sodium salts.

B) copolymers of acrylic acid or methacrylic acid and of a monoethylenic monomer, such as ethylene, styrene, vinyl esters, acrylic or methacrylic acid esters.

These copolymers may be grafted onto a polyalkylene glycol, such as polyethylene glycol and are optionally crosslinked.

Such polymers are especially described in the French patent application FR 1,222,944 and in the German patent application DE 2,330,956. Copolymers may be especially mentioned containing in their chain an optionally N-alkylated and/or hydroxyalkylated acrylamide unit, such as those described in the Luxemburger patent applications LU 75370 and LU75371 or sold under the trade name QUADRAMER® by AMERICAN CYANAMID.

Copolymers of acrylic acid and C₁-C₄ alkyl methacrylate and terpolymers of vinyl pyrrolidone, (meth)acrylic acid and C₁-C₂₀ alkyl (meth)acrylate may also be mentioned, for example of lauryl (ACRYLDONE® LM marketed by ISP), of tert-butyl (LUVIFLEX® VBM 70 marketed by BASF) or of methyl (STEPANHOLD® EXTRA marketed by STEPAN), and terpolymers of methacrylic acid/ethyl acrylate/tert-butyl acrylate, such as the product marketed under the trade name LUVIMER® 100 P by BASF, and terpolymers polyvinyl pyrrolidone/vinyl acetate/vinyl propionate, especially those marketed under the trade name Luviskol VAP 343 by BASF.

C) crotonic acid derived copolymers, such as those containing in their chain vinyl acetate or propionate units and optionally other monomers, such as allylic, methallylic or vinylic esters of a saturated, linear or branched, hydrocarbon long-chain carboxylic acid with at least 5 carbon atoms, these polymers being optionally grafted and crosslinked, or vinyl, allyl or methallyl esters of a α- or β-cyclic carboxylic acid.

Such polymers are described, inter alia, in the French patent applications FR 1,222,944, FR 1,580,545, FR 2,265,782, FR 2,265,781, FR 1,564,110 and FR 2,439,798.

There can be mentioned as examples of trade products belonging to this category 28-29-30, 26-13-14 and 28-13-10 resins marketed by NATIONAL STARCH.

D) carboxylic acid or anhydride derived, C₄-C₈ monounsaturated copolymers selected from:
copolymers comprising:

(i) one or more maleic, fumaric, itaconic acid(s) or anhydride(s) and

(ii) at least one monomer selected from vinyl esters, vinyl ethers, vinyl halides, phenyl vinyl derivatives, acrylic acid and esters thereof, the anhydride functionalities of these copolymers being optionally monoesterified or monoamidified.

Such polymers are especially described in the patent applications U.S. Pat. No. 2,047,398, U.S. Pat. No. 2,723,248, U.S. Pat. No. 2,102,113 and GB 839,805 and especially those marketed under the trade names GANTREZ AN or ES, AVANTAGE® CP by ISP;
copolymers comprising

(iii) one or more maleic, citraconic or itaconic anhydride(s) and

(iv) one or more monomer(s) selected from allyl or methallyl esters containing optionally in their chain one or more acrylamide, methacrylamide, α-olefin, acrylic or methacrylic ester, acrylic or methacrylic acid or vinyl pyrrolidone moieties, the anhydride functionalities of these copolymers being optionally monoesterified or monoamidified.

These polymers are for example described in the French patent applications FR 2,350,384 and FR 2,357,241.

E) carboxylate moiety containing polyacrylamides.

Anionic polymer anionic moieties of the present invention may also be sulfonic acid groups provided by vinyl sulfonic, styrene sulfonic, naphthalene sulfonic or acrylamidoalkyl sulfonic moieties.

These sulfonic acid group containing polymers are especially selected from:

poly(vinyl sulfonic acid) salts having a weight average molecular weight ranging from about 1000 to 100 000, as well as copolymers of vinyl sulfonic acid and of a unsaturated comonomer, such as acrylic acid, methacrylic acid, esters of these acids, acrylamide, acrylamide derivatives, vinyl ethers and vinyl pyrrolidone;

poly(styrene sulfonic acid) salts. A suitable example includes two sodium salts with a weight average molecular weight ranging from about 500 000 to about 100 000 respectively marketed under the trade names FLEXAN® 500 and FLEXAN® 130 by NATIONAL STARCH. These compounds are described in the French patent FR 2,198,719;

poly(acrylamide sulfonic acid) salts, such as those mentioned in the American patent U.S. Pat. No. 4,128,631 and more particularly the poly(acrylamidoethyl propanesulfonic acid) marketed under the trade name COSMEDIA POLYMER® HSP 1180 by HENKEL.

According to the invention, anionic polymers are preferably selected from acrylic acid copolymers, such as terpolymers of acrylic acid/ethyl acrylate/N-tert-butylacrylamide especially marketed under the trade name ULTRAHOLD STRONG® by BASF, crotonic acid derived copolymers, such as terpolymers of vinyl acetate/vinyl tert.-butyl benzoate/crotonic acid and terpolymers of crotonic acid/vinyl acetate/vinyl neododecanoate especially marketed under the trade name 28-29-30 resin by NATIONAL STARCH, maleic, fumaric or itaconic acid or anhydride-derived copolymers containing as comonomers vinyl esters, vinyl ethers, vinyl halides, phenylvinyl derivatives, acrylic acid and acrylic acid esters, such as methylvinyl ether/monoesterified maleic anhydride copolymers, marketed for example under the trade name GANTREZ® by ISP, methyl methacrylate acid copolymers marketed under the trade name EUDRAGIT®L by ROHM PHARMA, copolymers of methacrylic acid/methyl methacrylate/C₁₋₄ alkyl acrylate /acrylic acid or C₁₋₄ hydroxyalkyl methacrylate, marketed under the trade name AMERHOLD® DR 25 by AMERCHOL, or under the trade name ACUDYNE® 255 by ROHM & HAAS, methacrylic acid and ethyl acrylate copolymers marketed under the trade name LUVIMER® MAEX or MAE by BASF and vinyl acetate/crotonic acid copolymers and vinyl acetate/crotonic acid copolymers grafted with polyethylene glycol marketed under the trade name ARISTOFLEX® A by BASF.

Amphoteric polymers suitable for use in the present invention are especially selected from polymers containing B and C units statistically distributed in the polymer chain, where B represents a unit derived from a monomer containing at least one basic nitrogen atom and C represents a unit derived from a monomer containing one or more carboxylic acid or sulfonic acid moieties. Amphoteric polymers may also comprise zwitterionic units of the carboxybetaine or sulfobetaine type. There can be cationic main chain polymers as well, containing primary, secondary, tertiary or quaternary amine moieties, amongst which at least one, bears—through a hydrocarbon radical—a carboxylic acid or a sulfonic acid moiety. Amphoteric polymers may further have an anionic chain derived from dicarboxylic α,β-unsaturated acids, one of the carbonyl moieties of which was brought to react with a polyamine containing one or more primary or secondary amine moieties.

Amphoteric polymers corresponding to the definition given hereabove are especially selected from the following polymers:

(1) polymers resulting from copolymerization of a carboxylic acid moiety bearing vinyl monomer, such as acrylic acid, methacrylic acid, maleic acid, α-chloroacrylic acid, and of a vinyl monomer containing at least one basic functionality, such as dialkylaminoalkyl methacrylate and acrylate or dialkylaminoalkyl (meth)acrylamides. Such compounds are described for example in the American patent application U.S. Pat. No. 3,836,537.

(2) Polymers containing units derived from:
(a) at least one monomer selected from N-alkylated acrylamides or methacrylamides,
(b) at least one comonomer containing one or more carboxylic acid functionalities, and
(c) at least one basic comonomer, such as primary, secondary, tertiary or quaternary amine substituted, acrylic acid and methacrylic acid esters and the quaternization product of dimethylaminoethyl methacrylate with dimethyl or diethyl sulfate.

N-alkylated (a) preferred acrylamides or methacrylamides are those bearing C₂₋₁₂alkyl radicals, such as N-ethylacrylamide, N-tert-butylacrylamide, N-tert.-octylacrylamide, N-octylacrylamide, N-decylacrylamide, N-dodecylacrylamide, as well as corresponding methacrylamides.

Carboxylic acid group containing comonomers (b) are more particularly selected from acrylic, methacrylic, crotonic, itaconic, maleic, fumaric acids, as well as from maleic or fumaric acid or anhydride C₁₋₄ alkyl monoesters.

Preferred basic comonomers (c) are aminoethyl methacrylate, butylaminoethyl methacrylate, N,N′-dimethylaminoethyl methacrylate and N-tert-butylaminoethyl methacrylate.

Copolymers are especially used, the CTFA denomination of which (4^{th} Ed., 1991) is “Octylacrylamide/acrylates/butylaminoethyl-meth-acrylate copolymer”, such as the products marketed under the trade name AMPHOMER® or LOVOCRYL® 47 by NATIONAL STARCH.

(3) crosslinked and alkylated polyaminoamides, fully or partly derived from polyaminoamides of general formula (5):
—[C(=0)—R₁₄—C(=0)—Z—]— (5)
wherein R₁₄ represents a divalent radical derived from a saturated dicarboxylic acid, a mono- or dicarboxylic aliphatic acid with an ethylene double bond, a C₁₋₆ alkyl ester of these acids or a radical resulting from the addition of any one of said acids on a bis primary or bis secondary amine, and Z represents a polyalkylene- bis primary, mono- or bis secondary polyamine radical, and preferably represents:

a) in amounts ranging from 60 to 100% by mole, the radical of formula (6):
—NH—[(CH₂)ₓ—NH]ₚ— (6)
wherein x=1 and p=2 or 3, or x=3 and p=2,
this radical being derived from diethylenetriamine, triethylenetetraamine or dipropylenetriamine;

b) in amounts ranging from 0 to 40% by mole, the radical of formula (III) wherein x=2 and p=1, derived from ethylenediamine, or the radical 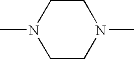
derived from piperazine:

c) in amounts ranging from 0 to 20% by mole, the radical —NH—(CH₂)₆—NH— derived from hexamethylenediamine, these polyaminoamines being crosslinked by adding from 0.025 to 0.35 mole by mole of the amine moiety, of a difunctional crosslinking agent selected from epihalohydrines, diepoxides, dianhydrides, di-unsaturated compounds, and alkylated with acrylic acid, chloroacetic acid or an alkane sulfone.

Saturated carboxylic acids are preferably selected from acids containing from 6 to 10 carbon atoms, such as adipic acid, trimethyl-2,2-4-adipic acid and trimethyl-2,4,4-adipic acid, terephthalic acid, ethylene double bond acids, such as for example acrylic, methacrylic and itaconic acids.

Alkane sulfones used for the alkylation are preferably propane sulfone or butane sulfone.

Alkylating agent salts are preferably sodium or potassium salts.

(4) Polymers containing zwitterionic units of formula (7): 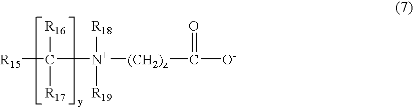
wherein R₁₅ represents a polymerizable unsaturated moiety, such as an acrylate, methacrylate, acrylamide or methacrylamide moiety, y and z each represent an integer between 1 and 3, R₁₆ and R₁₇ each represent independently a hydrogen atom or a methyl, ethyl or propyl group, R₁₈ and R₁₉ each represent independently a hydrogen atom or an alkyl radical, the atom carbon total number in R₁₈ and R₁₉ not exceeding 10.

Moreover, polymers comprising such units of formula (VII) may comprise non zwitterionic monomer derived units, such as dimethyl or diethylaminoethyl acrylate or methacrylate, alkyl acrylates or methacrylates, acrylamides or methacrylamides or vinyl acetate.

As an example, methyl methacrylate/ dimethyl carboxymethyl ammonioethyl methacrylate copolymer may be mentioned, such as the product marketed under the trade name DIAFORMER® Z301 by SANDOZ.
(5) Chitosan derived polymers containing monomer units corresponding to the following formulas:

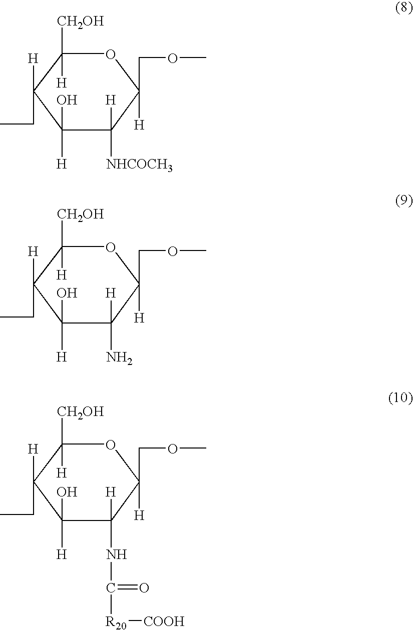

the unit of formula (8) being present in amounts ranging from 0 to 30%, the unit of formula (9) in amounts ranging from 5 to 50% and the unit of formula (10) in amounts ranging from 30 to 90%, being understood that in this unit (10), R₂₀ represents a radical of formula (11): 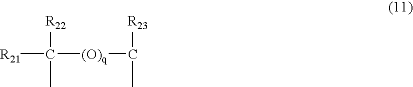
wherein:

if q=0, then R₂₁, R₂₂ and R₂₃, which may be the same or different, each represent a hydrogen atom, a methyl, hydroxyl, acetoxy or amino group, a monoalkyl amine or dialkyl amine group optionally interrupted by one or more nitrogen atom(s) and/or optionally substituted by one or more amine, hydroxyl, carboxyl, alkylthio or sulfo group(s), an alkylthio group, the alkyl group of which bears an amino residue, at least one of the R₂₁, R₂₂ and R₂₃ radicals being in this case a hydrogen atom; or if q=1, then R₂₁, R₂₂ and R₂₃ each represent a hydrogen atom, as well as salts formed by these compounds with bases or acids.

6) Polymers obtained by chitosan N-carboxyalkylation, such as N-carboxymethyl chitosan or N-carboxybutyl chitosan marketed under the trade name EVALSAN® by JAN DEKKER.

(7) Polymers corresponding to the following general formula (12): 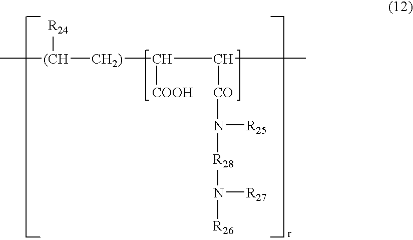
especially described in the French patent application FR 1,400,366, wherein R₂₄ represents a hydrogen atom or a CH₃O, CH₃CH₂O or phenyl radical, R₂₅ represents a hydrogen atom or a lower alkyl radical, such as methyl or ethyl, R₂₆ represents a hydrogen atom or a lower alkyl radical, such as methyl or ethyl, R₂₇ represents a lower alkyl radical, such as methyl or ethyl or a radical corresponding to the following formula:
—R₂₈—N(R₂₂)₂,

R₂₈ representing a —CH₂—CH₂—, —CH₂—CH₂—CH₂—,

—CH₂—CH(CH₃)-moiety and R₂₂ being as defined hereabove, as well as higher homologues of these radicals containing up to 6 carbon atoms.

(8) Amphoteric polymers of -D-X-D-X- type selected from:
(a) polymers obtained by the effect of chloroacetic acid or sodium chloroacetate on compounds containing at least one unit of formula:
   -D-X-D-X-D- (13)
   where D represents a radical: 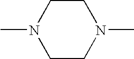
   and X represents the symbol E or E′, which may be the same or different, representing a divalent radical which is a linear or branched chain alkylene radical containing up to 7 carbon atoms in the main chain unsubstituted or substituted by hydroxyl moieties and that may further comprise oxygen, nitrogen or sulfur atoms, from 1 to 3 aromatic and/or heterocyclic ring(s), oxygen, nitrogen and sulfur atoms being present as ether, thioether, sulfoxide, sulfone, sulfonium, alkyl amine, alkenyl amine moieties, hydroxyl, benzylamine, amine oxide, quaternary ammonium, amide, imide, alcohol, ester and/or urethane moieties.

b) Polymers of formula:
-D-X′-D-X′-
wherein D represents a radical 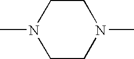
and X′ represents the symbol E or E′ and at least once E′, E being as defined hereabove and E′ being a divalent radical which is a linear or branched alkylene radical with up to 7 carbon atoms in the main chain, substituted or not by one or more hydroxyl radical(s) and containing one or more nitrogen atom(s), the nitrogen atom being substituted by an alkyl chain that is optionally interrupted by an oxygen atom and compulsory containing one or more carboxyl functionalities or one or more hydroxyl functionalities and betainized by reacting with chloroacetic acid or with sodium chloroacetate.

9) (C₁₋₅)alkyl vinylether/maleic anhydride copolymers partly modified by half-amidification with a N,N-dialkylaminoalkyl amine, such as N,N-dimethyl aminopropyl amine, or by half-esterification with a N,N-dialkanol amine. These copolymers may also comprise other vinyl comonomers, such as vinyl caprolactame.

Amphoteric polymers preferred according to the invention are those of family (3) described hereabove, such as those having following CTFA denomination “Octylacryl amide/acrylates/butyl aminoethyl methacrylate copolymer”. The products marketed under the trade names AMPHOMER®, AMPHOMER® LV 71 or LOVOCRYL® 47 by NATIONAL STARCH may be mentioned as examples.

Other preferred amphoteric polymers are those of family (4), as for example methyl methacrylate and dimethylcarboxymethyl ammonioethyl methacrylate copolymers, marketed for example under the trade name DIAFORMER® Z301 by SANDOZ.

Anionic or amphoteric polymers, if needed, may be partly or fully neutralized. Neutralizing agents are for example soda, potash, amino-2-methyl propanol, monoethanolamine, triethanolamine or triisopropanolamine, inorganic or organic acids, such as hydrochloric acid or citric acid.

Non ionic polymers that can be used according to the invention are selected for example from:

vinyl pyrrolidone homopolymers,

vinyl pyrrolidone and vinyl acetate copolymers,

polyalkyl oxazolines, such as polyethyl oxazolines marketed by DOW CHEMICAL under the trade names PEOX® 50 000, PEOX® 200 000 and PEOX® 500 000,

vinyl acetate homopolymers, such as the product marketed under the trade name APPRETAN® EM by HOECHST or the product marketed under the trade name RHODOPAS® A 012 by RHONE POULENC;

vinyl acetate and acrylic ester copolymers, such as the product marketed under the trade name RHODOPAS® AD 310 by RHONE POULENC,

vinyl acetate and ethylene copolymers, such as the product marketed under the trade name APPRETAN® TV by HOECHST,

vinyl acetate and maleic ester copolymers for example dibutyl maleate-based copolymers, such as the product marketed under the trade name APPRETAN® MB EXTRA by HOECHST,

ethylene and maleic anhydride copolymers,

alkyl acrylate homopolymers and alkyl methacrylate homopolymers, such as the product marketed under the trade name MICROPEARL® RQ 750 by MATSUMOTO or the product marketed under the trade name LUHYDRAN® A 848 S by BASF,

acrylic ester copolymers, such as for example alkyl acrylate and alkyl methacrylate copolymers, such as the products marketed by ROHM & HAAS under the trade names PRIMAL AC-261 K and EUDRAGIT NE 30 D, by BASF under the trade names ACRONAL® 601, LUHYDRAN® LR 8833 or 8845, and by HOECHST under the trade names APPRETAN® N 9213 or N 9212,

copolymers of acrylonitrile and a non ionic monomer selected for example from butadiene and alkyl (meth)acrylates; products can be mentioned, as those marketed under the trade names NIPOL® LX 531 B by NIPPON ZEON or those marketed under the trade name CJ 0610 B by ROHM & HAAS,

polyurethanes, such as the products marketed under the trade names ACRYSOL® RM 1020 or ACRYSOL® RM 2020 by ROHM & HAAS, the products URAFLEX® XP 401 UZ, URAFLEX® XP and 402 UZ by DSM RESINS,

alkyl acrylate and urethane copolymers, such as the product 8538-33 marketed by NATIONAL STARCH,

polyamides, such as the product ESTAPOR® LO 11 marketed by RHONE POULENC,

chemically modified or not, non ionic guar gums,

polyvinyl alcohols, such as MOWIOL® 40-88 marketed by HOECHST.

Non modified, non ionic guar gums are for example the products marketed under the trade name VIDOGUM® GH 175 by UNIPECTINE and under the trade name JAGUAR® C by MEYHALL. Modified non ionic guar gums suitable for use according to the invention are preferably modified by C₁₋₈ hydroxyalkyl moieties. There can be mentioned as examples hydroxymethyl, hydroxyethyl, hydroxypropyl and hydroxybutyl moieties.

These guar gums are well known in the art and may for example be prepared by reacting corresponding alkene oxides, such as for example propylene oxide with the guar gum so as to produce a hydroxypropyl moiety modified guar gum.

Such non ionic guar gums optionally modified by hydroxyalkyl moieties are for example marketed under the trade names JAGUAR® HP8, JAGUAR® HP60 and JAGUAR® HP120, JAGUAR® DC 293 and JAGUAR® HP 105 by MEYHALL, or under the trade name GALACTASOL® 4H4FD2 by AQUALON.

The foaming agent(s) represent(s) from 0.01 to 20%, preferably from 0.05 to 10%, by weight as compared to the total weight of the cosmetic composition.

Amongst alkanols that can be used according to the invention, there are C₁-C₆ alkanols, such as ethanol, propanol, butanol or a mixture thereof. Ethanol is the most preferred alkanol.

The alkanol(s) generally represent(s) up to 20%, preferably up to 15% by weight alkanol as compared to the total weight of the cosmetic composition.

The aqueous phase according to the invention may further comprise pearling agents.

As used herein, a pearling agent means iridescent particles that reflect light.

Such pearling agents may be selected from white perlescent pigments, such as titanium-coated mica or bismuth oxichloride pigments, coloured perlescent pigments, such as mica-titanium with iron oxides, mica-titanium with in particular iron blue or chromium oxide, mica-titanium with an organic pigment of the aforementioned type.

In order to improve hair fiber cosmetic properties or also to reduce or to prevent their damaging, the composition used according to the invention may further comprise one or more cosmetic additive(s).

Such additive(s) is or are usually selected from hair fiber protecting agents, vitamins or provitamins, natural waxes, ceramides, proteins, fragrances, preserving agents, sequestering agents, acidifying agents, alkalinizing agents, as well as their mixtures.

The above cosmetic additive(s) usually represent(s) from 0.001 to 20%, preferably from 0.1 to 10% by weight as compared to the total weight of the composition.

The spray compositions according to the invention may further comprise silicones.

These silicones may be volatile or not, linear or cyclic silicones.

Silicones suitable for use according to the present invention are especially polyorganosiloxanes and may come as oils, waxes, resins or gums.

Organopolysiloxanes are defined in more detail in “Chemistry and Technology of Silicones” Walter NOLL (1968) Academic Press. They may be volatile or not.

Non volatile silicones are preferably used and more particularly polyalkyl siloxanes, polyaryl siloxanes, polyalkylaryl siloxanes, silicone gums and resins, organofunctional moiety modified polyorganosiloxanes, as well as their mixtures.

Silicone gums suitable for use according to the present invention are especially polydiorganosiloxanes having high number average molecular weights ranging from 200 000 to 1 000 000 used either alone or in admixture in a solvent. This solvent may be selected from volatile silicones, polydimethyl siloxane oils (PDMS), polyphenylmethyl siloxane oils (PPMS), isoparaffins, polyisobutylenes, methylene chloride, pentane, dodecane, tridecane or their mixtures.

Following products may be especially mentioned:

polydimethyl siloxane

polydimethyl siloxane/methylvinyl siloxane gums,

polydimethyl siloxane/diphenyl siloxane,

polydimethyl siloxane/phenylmethyl siloxane,

polydimethyl siloxane/diphenyl siloxane/methylvinyl siloxane.

Organopolysiloxane resins suitable for use according to the present invention are crosslinked siloxane systems comprising following units:

R′₂SiO_{2/2}, R′₃SiO_{1/2}, R′SiO_{3/2} and SiO_{4/2} wherein R′ represents a hydrocarbon moiety containing from 1 to 16 carbon atom(s) or a phenyl moiety. Amongst these products, the most preferred are those where R represents a lower C₁-C₄ alkyl radical, more particularly a methyl or a phenyl radical.

Organomodified silicones suitable for use according to the present invention are silicones such as defined hereabove that comprise in their structure one or more organofunctional moieties bound with a hydrocarbon radical.

Especially mentioned as organomodified silicones are polyorganosiloxanes containing:

polyethyleneoxy and/or polypropyleneoxy moieties containing optionally C₆-C₂₄ alkyl moieties, such as the products called dimethicone copolyol marketed by DOW CORNING under the trade name DC 1248 or the SILWET L 722, L 7500, L 77, L 711 oils marketed by UNION CARBIDE and (C₁₂)-alkyl methicone copolyol marketed by DOW CORNING under the trade name Q2 5200;

Substituted or not, amine moieties, such as the products marketed under the trade name GP 4 Silicone Fluid and GP 7100 by GENESEE or the products marketed under the trade names Q2 8220 and DOW CORNING 929 or 939 by DOW CORNING. Substituted amine moieties are especially C₁-C₄-aminoalkyl moieties;

thiol moieties such as the products marketed under the trade names “GP 72 A” and “GP 71” by GENESEE;

alkoxylated moieties such as the product marketed under the trade name “SILICONE COPOLYMER F-755” by SWS SILICONES and ABIL WAX 2428, 2434 and 2440 by GOLDSCHMIDT;

hydroxylated moieties, such as hydroxyalkyl functionality polyorganosiloxanes described in the French patent application FR-A-85 16334 corresponding to the following formula (15):

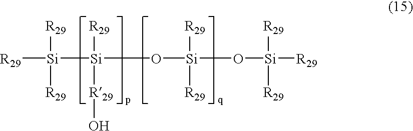

wherein R₂₉ radicals, which may be the same or different, are selected from methyl and phenyl radicals; at least 60% by mole R₂₉ radicals representing methyl; the R′₂₉ radical is a divalent hydrocarbon C₂-C₁₈ alkylene chain member; p is between 1 and 30 inclusive; q is between 1 and 150 inclusive;

acyloxyalkyl moieties, such as for example polyorganosiloxanes described in the American patent U.S. Pat. No. 4,957,732 and corresponding to the following formula (16):

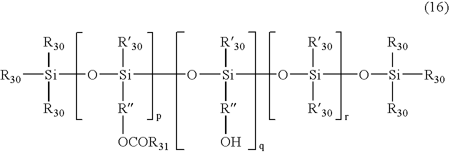

wherein:

R₃₀ represents a methyl, phenyl, —OCOR₃, hydroxyl moiety and only one of the R₃₀ radicals per silicon atom can be OH;

R′₃₀ represents a methyl, phenyl; at least 60% by mole as compared to the totality of the R₃₀ and R′₃₀ radicals representing a methyl;

R₃₁ represents a C₈-C₂₀ alkyl or alkenyl;

R″ represents a linear or branched divalent hydrocarbon C₂-C₁₈ alkylene radical;

r is between 1 and 120 inclusive;

p is between 1 and 30;

q is 0 or is lower than 0.5 p, p +q is between 1 and 30; polyorganosiloxanes of formula (16) may comprise following moieties:

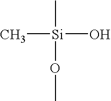

in amounts that do not exceed 15% of p+q+r.

carboxylic type anionic moieties, as for example in the products described in the patent EP 186 507 and marketed by CHISSO CORPORATION, or of alkylcarboxylic type, such as those present in the product X-22-3701E marketed by SHIN-ETSU; 2-hydroxyalkyl sulfonate; 2-hydroxyalkyl thiosulfate, such as the products marketed by GOLDSCHMIDT under the trade names “ABIL S201” and “ABIL S255”.

hydroxyacylamino moieties, such as polyorganosiloxanes described in the application EP 342 834. As an example, the product Q2-8413 marketed by DOW CORNING may be mentioned.

Particularly preferred polyorganosiloxanes according to the present invention are polyorganosiloxanes containing polyethyleneoxy and/or polypropyleneoxy moieties optionally with C₆-C₂₄ alkyl moieties, such as the product called dimethicone copolyol marketed by DOW CORNING under the trade name DC 1248 or SILWET L 722, L 7500, L 77, L 711 oils marketed by UNION CARBIDE and (C₁₂)alkyl methicone copolyol marketed by DOW CORNING under the trade name Q2 5200.

According to the invention, all silicones may also be used in a soluble, dispersed, or microdispersed form, as emulsions, nanoemulsions or microemulsions.

In addition to or rather than alkanols, the composition may comprise one or more cosmetically acceptable solvent(s), preferably selected from polyols, such as glycerol, propylene glycol, pentanediol, and benzyl alcohol.

This or these cosmetically acceptable solvent(s) according to the invention represent(s) from 0.001 to 20% by weight as compared to the total weight of the composition, preferably from 0.1 to 10% by weight of the composition.

Hair fiber protecting agents may be any active agent useful for preventing or restraining damages resulting from physical or chemical aggressions.

So, the hair fiber protecting agent may be selected from organic water-soluble, liposoluble UV filters, free-radical scavengers, antioxydants, vitamins, provitamins, as well as their mixtures.

Organic UV filters (UV radiation filtering systems) are especially selected from silicone or non silicone, water-soluble or liposoluble filters, and mineral oxide nanoparticles, the surface of which has been optionally treated so as to make it hydrophilic or hydrophobic.

Water-soluble, organic UV filters may be selected for example from para-aminobenzoic acid and salts thereof, anthranilic acid and salts thereof, salicylic acid and salts thereof, p-hydroxycinnamic acid and salts thereof, benz-x-azole sulfonic derivatives (benzothioazoles, benzimidazoles, benzoxazoles) and salts thereof, benzophenone sulfonic derivatives and salts thereof, benzylidene-camphor sulfonic derivatives and salts thereof, benzylidene-camphor derivatives substituted by a quaternary amine and salts thereof, phthalydene-camphosulfonic acid derivatives and salts thereof, benzotriazole sulfonic derivatives.

Hydrophilic polymers can also be used that have moreover anti-UV radiation photoprotective properties due to their chemical nature. Polymers containing benzylidene-camphor and/or benzotriazole moieties, substituted by sulfonic or quaternary ammonium moieties, can also be mentioned.

Liposoluble (or lipophilic) organic UV filters suitable for implementing in the present invention also especially include p-aminobenzoic acid derivatives, such as p-aminobenzoic acid esters or amides; salicylic acid derivatives, such as esters; benzophenone derivatives; dibenzoylmethane derivatives; diphenyl acrylate derivatives; benzofurane derivatives; polymeric UV filters containing one or more silico-organic residues; cinnamic acid esters; camphor derivatives; trianilino-s-triazine derivatives; urocanic acid ethyl ester; benzotriazoles; hydroxyphenyl triazine derivatives; bis-resorcinol dialkyl aminotriazine; and mixtures thereof.

The liposoluble (or lipophilic) UV filter according to the invention is preferably selected from: salicylate octyl; 4-tert-butyl-4′-methoxydibenzoyl methane (PARSOL 1789 ex GIVAUDAN); octocrylene; 2-ethylhexyl 4-methoxycinnamate (PARSOL MCX) and the compound of following formula (17), or 2-(2H-benzotriazol-2-yl)-4-methyl-6-[2-methyl-3-[1,3,3,3-tetramethyl-1-[(trimethylsilyl)oxy] disiloxanyl]propynyl]phenol, described in the patent application EP-A-0,392,883:

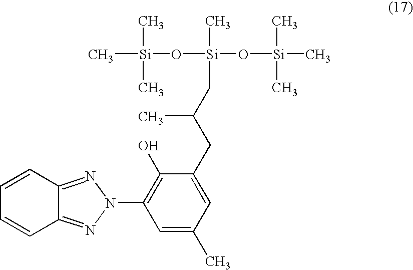

Other especially prefered UV filters according to the invention are benzophenone derivatives, such as UVINUL MS 40 (2-hydroxy 4-methoxybenzophenone-5-sulfonic acid) and UVINUL M40 (2-hydroxy-4-methoxybenzophenone) marketed by BASF, benzalmalonates derivatives, such as PARSOL SLX (polydimethyl/methyl (3(4-(2,2-bis-ethoxycarbonylvinyl)-phenoxy)-propenyl)siloxane) marketed by GIVAUDAN-ROURE, benzylidene-camphor derivatives, such as MEXORYL SX (b-b′camphosulfonic acid [1-4 divinylbenzene]) marketed by CHIMEX, benzimidazole derivatives, such as EUSOLEX 232 (2-phenyl-benzimidazol-5-sulfonic acid) marketed by MERCK.

The pH may be adjusted to the desired value by means of acidifying or alkalinizing agents as is well known in the art.

Suitable examples of alkalinizing agents comprise ammonia, alkaline carbonates, alkanolamines, such as mono-, di- and triethanolamines, as well as derivatives thereof, hydroxyalkyl amines and oxyethylene and/or oxypropylene ethylenediamines, sodium or potassium hydroxides and compounds of following formula (18): 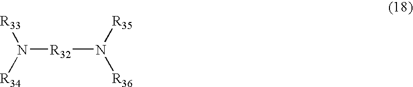
wherein R₃₂ is a propylene residue, optionally substituted by a hydroxyl moiety or a C₁-C₄ alkyl radical; R₃₃, R₃₄, R₃₅ and R₃₆, which may be the same or different, represent a hydrogen atom, a C₁-C₄ alkyl radical a or C₁-C₄ hydroxyalkyl radical.

Traditionally, acidifying agents are for example inorganic or organic acids such as hydrochloric acid, orthophosphoric acid, carboxylic acids such as tartaric acid, citric acid, lactic acid, or sulfonic acids.

As previously stated, the pressurized device, especially an aerosol, for hair styling et/or hair care according to the invention comprises a composition release device.

The pressurized composition release device, which forms part of the aerosol can, usually consists in a push-button activated distribution valve, itself comprising a diffuser through which the aerosol composition is released as a foam.

Preferably, the composition is released as a foam having a density as compared to water reaching up to 0.1.

The composition organic phase contained in the pressurized device representing the object of the invention further comprises a propellant selected from hydrocarbons, such as n-butane, propane, isobutane, pentane, chlorinated and/or fluorinated hydrocarbons, and mixtures thereof.

1,1-difluoroethane is one of the preferred halocarbons.

Since the organic phase of the composition is pressurized in the container it is usually contained therein in a liquid form.

The selected hydrocarbon is preferably isobutane.

Pressurized device pressure at 20° C. will be especially regulated according to the nature of the hydrocarbon(s) used.

Propellant(s) usually represent(s) up to 25%, preferably from 2 to 15% and more preferably from 5 to 12% by weight as compared to the total weight of the composition, as described hereabove.

An embodiment of the pressurized device of the invention is illustrated in the accompanying drawings and will be described hereafter as an only illustrative example that should not in any way be considered as limitative.

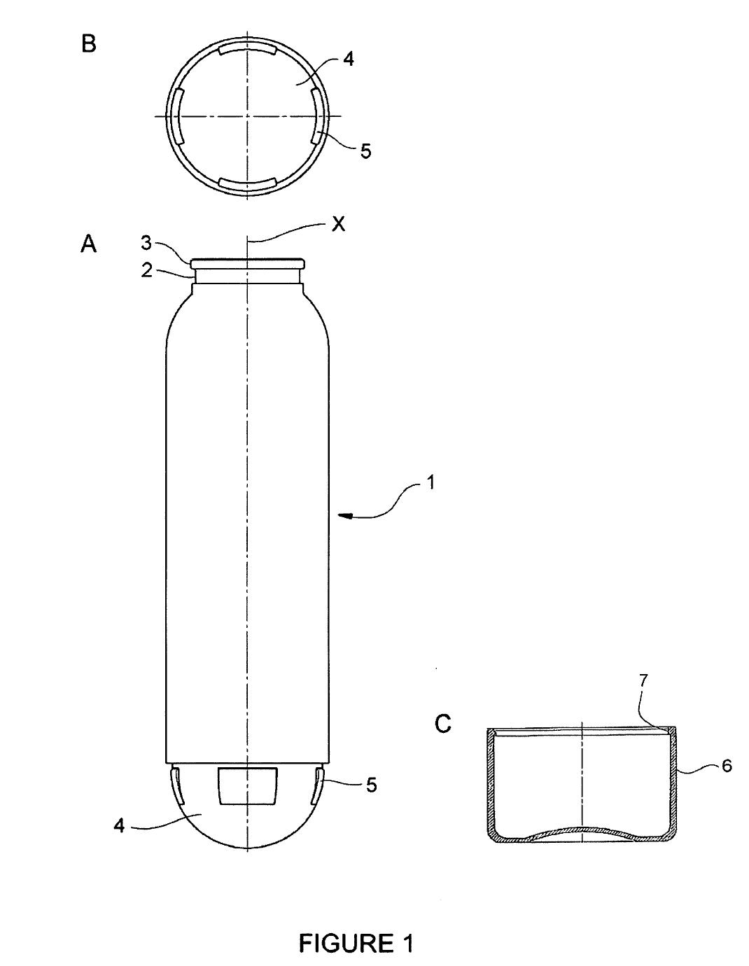

FIG. 1A represents an axial view of an aerosol can according to the invention, in the opened position.

FIG. 1B represents an underneath view of the container.

FIG. 1C represents a cross-section view of the reinforcement cup casing.

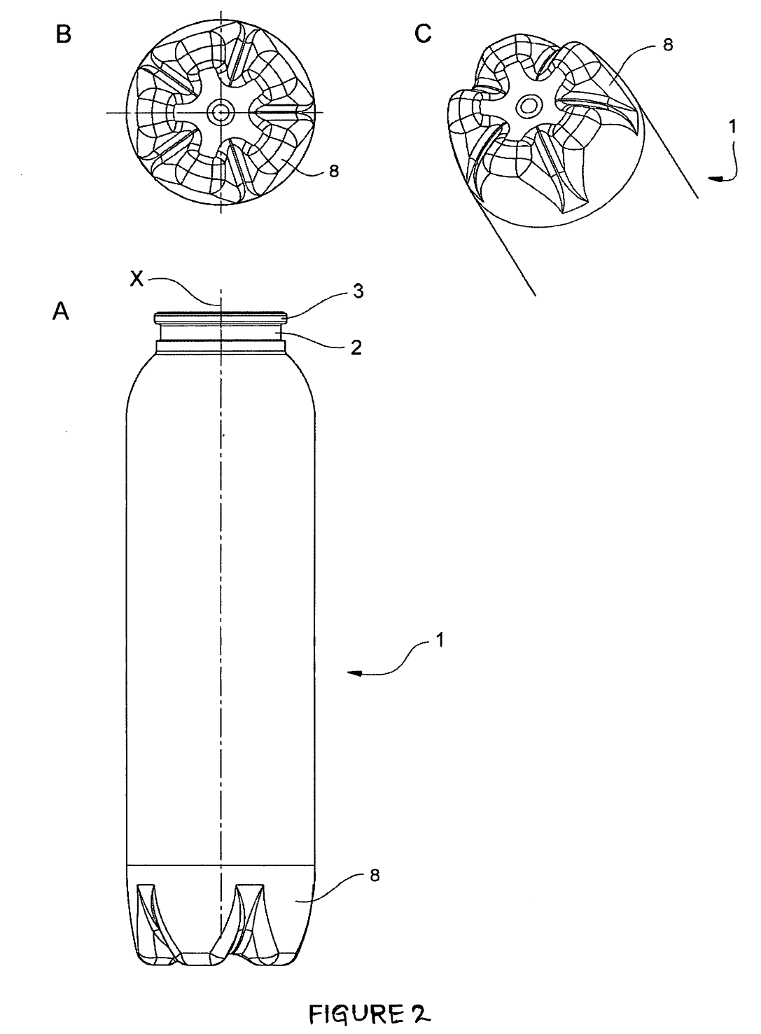

FIG. 2A represents an alternative embodiment of the container illustrated in an axial plane and comprising a concave-shaped bottom.

FIG. 2B represents an underneath view of the concave-shaped bottom.

FIG. 2C represents an isometric view of the bottom as illustrated in FIGS. 2A and 2B.

FIG. 1A shows a container hereafter identified with 1 as a whole.

Preferably, the container comprises at least one longitudinal axis X cylindrical portion, having an opened neck 2. It is understood that the container may have a slightly flattened or elongated shape according to axis X, that could be interesting with regard to the aesthetic point of view.

Preferably, device's container according to the invention is cylindrical on its whole height.

The cylindrical shape of this container enables especially the pressurized device according to the invention to be easily recognized or identified by consumers as a pressurized container hair styling product.

Moreover when many containers are packaged or brought together, those of cylindrical shape take up far less space than those of spherical shape, thus saving space when packaged or shown on display shelves.

The feed opening formed at the free end of neck 2 has a collar 3 to which a valve retainer (not illustrated) is usually fixed.

FIG. 1B illustrates the container comprising a reinforcement cup casing 6 placed on bottom 4.

The bottom 4 may have a convex hemispherical shape and the reinforcement cup casing 6 may be provided with a flange 7 so as to fix the cup casing on bottom 4. The hemispherical bottom may comprise lugs 5 to work together with the flange so as to maintain the cup casing 6 on bottom 4 of container 1 (FIG. 1C).

FIGS. 2A, 2B and 2C represent an alternative embodiment of the pressurized device according to the invention comprising a container having a concave bottom 8.

Preferably, the container comprises a bottom having a concavity.

According to an alternative embodiment, the container comprises a bottom having a plurality of concavities.

According to an alternative embodiment, the container according to the invention has a flat bottom.

Preferably, the container comprises a reinforcement cup casing placed on the bottom.

Preferably, container wall thickness (neck and bottom exclusive) ranges from 0.45 to 1 mm.

The test pressure resistance and the vacuum resistance of the container according to the invention have been especially determined.

As used herein, test pressure resistance means the pressure the empty container may be put under, for 25 seconds at 20° C. without any leakage or without any visible or permanent deformation occurs.

As used herein, vacuum resistance means the negative pressure the container may be put under, for 25 seconds at 20° C. without any air penetration or without any visible or permanent deformation occurs.

The container according to the invention demonstrated a test pressure resistance value higher than 20 bars, and a vacuum resistance value higher than 0.6 bar. The container according to the invention is usually formed by strech-blowing according to methods that are well known by the one skilled in the art.

It is a further object of the present invention to provide a hair fiber styling composition packaged in a pressurized container, such as previously described.

The present invention is illustrated by means of the following example:

### EXAMPLE

A hair styling and/or hair care foam aerosol is prepared by pressurizing in a container made of a combination of polyethylene naphthalate and polyethylene terephthalate (90:10) a two-phase cosmetic composition which comprises, by weight as compared to the total weight of the composition:

| | |
|---|---|
| Polyquaternium-30: | 0.55% |
| Polyquaternium-4: | 0.5% |
| Laureth-4: | 0.05% |
| Copolymer VC 713 (ISP): | 0.7% AM |
| Mowiol 40-88 (Hoechst): | 0.1% AM |
| Ethanol: | 8% |
| Fragrance: | 0.1% |
| Water: | 80% |
| Isobutane: | 10% |

AM: active material

The resulting aerosol product is released as a foam that can be easily applied onto the hair fibers to style the hair.

What is claimed is:

1. A pressurized device, especially an aerosol, comprising:
a) a container, the wall of which is made at least partly of a mixture comprising at least one polyethylene naphthalate and at least one polyethylene terephthalate,
b) a cosmetic composition comprising:
an aqueous phase comprising at least one foaming agent; and
an organic phase comprising at least one propellant selected from halogenated or not halogenated hydrocarbons and mixtures thereof, said device being able to withstand high temperatures.

2. A device according to claim 1, characterized in that the container a) is transparent or translucent.

3. A device according to claim 1 or 2, characterized in that the composition b) is transparent or translucent.

4. A device according to any one of preceding claims, characterized in that the container a) has at least one cylindrical portion.

5. A device according to claim 4, characterized in that the container a) is cylindrical on its whole height.

6. A device according to any one of preceding claims, characterized in that the container a) comprises a concave bottom.

7. A device according to claim 6, characterized in that the container a) comprises a bottom having a concavity.

8. A device according to claim 6, characterized in that the container a) comprises a bottom having a plurality of concavities

9. A device according to any one claims 1 to 5, characterized in that the container has a flat bottom.

10. A device according to any one of preceding claims, characterized in that the container comprises a reinforcement cup casing placed on the bottom.

11. A device according to any one of preceding claims, characterized in that the container comprises, by weight of the polymer total weight, from 60% to 95%, preferably 90% polyethylene naphthalate and from 5% to 40%, preferably 10% polyethylene terephthalate.

12. A device according to any one of preceding claims, characterized in that the foaming agent(s) is or are selected from cationic, anionic, non ionic, amphoteric or zwitterionic surfactants and cationic, anionic, non ionic, amphoteric or zwitterionic polymers, as well as mixtures thereof.

13. A device according to any one of preceding claims, characterized in that the foaming agent(s) represent(s) from 0.01 to 20%, preferably from 0.05 to 10%, by weight as compared to the total weight of the cosmetic composition.

14. Device according to any one of preceding claims, characterized in that the cosmetic composition comprises at least one alkanol selected from C₁- C₆ alkanols.

15. A device according to any one of preceding claims, characterized in that the cosmetic composition comprises up to 20%, preferably up to 15% by weight alkanol as compared to the total weight of the composition.

16. A device according to any one of preceding claims, characterized in that the cosmetic composition comprises at least one cosmetic additive selected from the group consisting of hair fiber protecting agents, vitamins or provitamins, natural waxes, ceramides, proteins, fragrances, preserving agents, sequestering agents, acidifying agents, alkalinizing agents and mixtures thereof.

17. A device according to any one of preceding claims, characterized in that the cosmetic additive(s) in the cosmetic composition represent(s) from 0.001% to 20%, preferably from 0.1 to 10% by weight as compared to the total weight of the composition.

18. A device according to any one of preceding claims, characterized in that the cosmetic composition comprises at least one volatile or not, linear or cyclic silicone.

19. A device according to claim 18 characterized in that at least one silicone is selected from polyorganosiloxanes

20. A device according to claim 19, characterized in that polyorganosiloxanes are selected from the group consisting of organofunctional moiety modified polyalkyl siloxanes, polyaryl siloxanes, polyalkylaryl siloxanes, polyorganosiloxanes, as well as their mixtures.

21. A device according to any one of claims 18 to 20, characterized in that silicones are also used in a soluble, dispersed, microdispersed form, as emulsions, nanoemulsions or microemulsions.

22. A device according to any one of preceding claims, characterized in that the cosmetic composition comprises at least one hair fiber protecting agent selected from the group consisting of water-soluble or liposoluble organic UV filters, free-radical scavengers, antioxydants, vitamins, provitamins, as well as their mixtures.

23. A device according to any one of preceding claims, characterized in that the cosmetic composition comprises one or more cosmetically acceptable solvent(s) selected from polyols, such as glycerol, propylene glycol, pentanediol and benzyl alcohol.

24. A device according to claim 23, characterized in that said cosmetically acceptable solvent(s) represent(s) from 0.001 to 20%, preferably from 0.1 to 10% as compared to the total weight of the cosmetic composition.

25. A device according to any one of preceding claims, characterized in that the propellant is selected from hydrocarbons, such as n-butane, propane, isobutane, pentane, chlorinated and/or fluorinated hydrocarbons, and mixtures thereof.

26. A device according to any one of preceding claims, characterized in that the propellant represents up to 30%, preferably from 2 to 15% and more preferably from 5 to 12% by weight as compared to the total weight of the cosmetic composition.

27. A device according to any one of preceding claims, characterized in that the selected propellant is isobutane.

28. A device according to any one of preceding claims, characterized in that the composition is released as a foam having a density as compared to water reaching up to 0.1.

29. A cosmetic composition comprising:
an aqueous phase comprising at least one foaming agent; and
an organic phase comprising at least one propellant selected from halogenated or not, hydrocarbons and mixtures thereof,
characterized in that it is packaged in a pressurized device, especially an aerosol, the wall of which is made at least partly of a mixture comprising at least one polyethylene naphthalate and at least one polyethylene terephthalate, said device being able to withstand high temperatures.

30. A cosmetic composition according to claim 29, characterized in that the container is transparent or translucent.

31. A cosmetic composition according to claims 29 or 32, characterized in that the composition is transparent or translucent.

32. A cosmetic composition according to any one of claims 29 to 31, characterized in that the container has at least one cylindrical portion.

33. A cosmetic composition according to any one of claims 29 to 32, characterized in that the container is cylindrical on its whole height.

34. A cosmetic composition according to any one of claims 29 to 33, characterized in that the container comprises a concave bottom.

35. A cosmetic composition according to claim 34, characterized in that the container comprises a bottom having a concavity.

36. A cosmetic composition according to claim 34, characterized in that the container comprises a bottom having a plurality of concavities.

37. A cosmetic composition according to any one of claims 29 to 34, characterized in that the container has a flat bottom.

38. A cosmetic composition according to any one of claims 29 to 37, characterized in that the container comprises a reinforcement cup casing placed on the bottom.

39. A cosmetic composition according to any one of claims 29 to 38, characterized in that the container comprises, by weight of the polymer total weight, from 60% to 95%, preferably 90% polyethylene naphthalate and from 5% to 40%, preferably 10% polyethylene terephthalate.

40. A cosmetic composition according to any one of claims 29 to 39, characterized in that the foaming agent(s) is or are selected from the group consisting of cationic, anionic, non ionic, amphoteric or zwitterionic surfactants and cationic, anionic, non ionic, amphoteric or zwitterionic polymers, as well as mixtures thereof.

41. A cosmetic composition according to any one of claims 29 to 40, characterized in that the foaming agent(s) represent(s) from 0.01 to 20%, preferably from 0.05 to 10%, by weight as compared to the total weight of the cosmetic composition.

42. A cosmetic composition according to any one of claims 29 to 41, characterized in that the cosmetic composition comprises at least one alkanol selected from C₁-C₆ alkanols.

43. A cosmetic composition according to any one of claims 29 to 42, characterized in that it comprises up to 20%, preferably up to 15% by weight alkanol as compared to the total weight of the composition.

44. A cosmetic composition according to any one of claims 29 to 43, characterized in that it comprises at least one cosmetic additive selected from the group consisting of hair fiber protecting agents, vitamins or provitamins, natural waxes, ceramides, proteins, fragrances, preserving agents, sequestering agents, acidifying agents, alkalinizing agents and mixtures thereof.

45. A cosmetic composition according to any one of claims 29 to 44, characterized in that the cosmetic additive(s) represent( )s in the cosmetic composition from 0.001% to 20%, preferably from 0.1 to 10% by weight as compared to the total weight of the composition.

46. A cosmetic composition according to any one of claims 29 to 45, characterized in that the cosmetic composition comprises at least one linear or cyclic, volatile or not silicone.

47. A cosmetic composition according to claim 46 characterized in that at least one silicone is selected from polyorganosiloxanes.

48. A cosmetic composition according to claim 47, characterized in that said polyorganosiloxanes are selected from organofunctional moiety modified polyalkyl siloxanes, polyaryl siloxanes, polyalkylaryl siloxanes, polyorganosiloxanes, as well as their mixtures.

49. A cosmetic composition according to any one of claims 46 to 48, characterized in that silicones are also used in a soluble, dispersed, or micro-dispersed form, as emulsions, as nanoemulsions or as microemulsions.

50. A cosmetic composition according to any one of claims 29 to 49, characterized in that it comprises at least one hair fiber protecting agent selected from the group consisting of water-soluble or liposoluble organic UV filters, free-radical scavengers, antioxydants, vitamins, provitamins, as well as their mixtures.

51. A cosmetic composition according to any one of claims 29 to 50, characterized in that it comprises one or more cosmetically acceptable solvent(s) selected from polyols, such as glycerol, propylene glycol, pentanediol and benzyl alcohol.

52. A cosmetic composition according to claim 51, characterized in that said cosmetically acceptable solvent(s) represent(s) from 0.001 to 20%, preferably from 0.1 to 10% by weight as compared to the total weight of the cosmetic composition.

53. A cosmetic composition according to any one of claims 29 to 52, characterized in that the propellant is selected from hydrocarbons, such as n-butane, propane, isobutane, pentane, chlorinated and/or fluorinated hydrocarbons and mixtures thereof.

54. A cosmetic composition according to any one of claims 29 to 53, characterized in that the propellant represents up to 30%, preferably from 2 to 15% and more preferably from 5 to 12% by weight as compared to the total weight of the cosmetic composition.

55. A cosmetic composition according to any one of claims 29 to 54, characterized in that the selected propellant is isobutane.

56. A cosmetic composition according to any one of claims 29 to 55, characterized in that it is released as a foam having a density as compared to water reaching up to 0.1.
